(19) United States
(12) Patent Application Publication
(10) Pub. No.: US 2023/0390365 A1
(43) Pub. Date: Dec. 7, 2023

(54) GENE THERAPEUTICS FOR ENHANCEMENT/RESTORATION OF ENDOMETRIAL FUNCTION

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/329,404

(22) Filed: Jun. 5, 2023

Related U.S. Application Data
(60) Provisional application No. 63/349,297, filed on Jun. 6, 2022.

(51) Int. Cl. A61K 38/44

(52) U.S. Cl. CPC A61K 38/44; C12Y 113/11052

(57) ABSTRACT

Disclosed are methods, compositions of matter and protocols useful for restoring/enhancing endometrial function by use of gene therapy. In one embodiment genes associated with endometrial preservation and/or regeneration are inserted into the endometrium and/or accessory tissues. Suppression of fibrosis, inflammation and stimulation of regeneration is accomplished by the practice of the current invention.

### CROSS-REFERENCE TO RELATED APPLICATIONS

This application claims priority to U.S. Provisional Application No. 63/349,297 titled “Gene Therapeutics for Enhancement/Restoration of Endometrial Function” filed Jun. 6, 2022, which is hereby incorporated by reference herein in its entirety.

### FIELD OF THE INVENTION

The invention relates to the use of gene therapeutics for regenerative treatment of the endometrium.

### BACKGROUND OF THE INVENTION

Studies have shown that more than 15% of women worldwide experience difficulties in becoming pregnant (WHO 1997), which is estimated to be 60 to 80 million women around the world a decade ago. Infertility is generally defined by the World Health Organization as a lack of conception after an arbitrary period of twelve months. However, many couples attempt for years to conceive naturally before seeking medical assistance in an effort to become pregnant. A decrease in fertility rate is associated with medical and non-medical factors. For example, women's age has been shown to be a direct major determinant of the average time required to conceive. It has been shown that premature ovarian failure occurs in 1:1,000 women before the age of 30; 1:250 women by 35 years; and 1:100 by the age of 40. Therefore, the highest birth rates are in the age group of 25-30 years and declines sharply after the age of 35 years. Infertility is currently one of the most frequent health concerns facing the population aged 25-45 years. Thus, great interest and need exist for a method of extending fertility in healthy women, possibly taking away age-related barriers to childbearing, and for women who are unable to conceive through natural methods. Although infertility itself may not threaten physical health, it often has a serious impact on the emotional, mental and spiritual well-being of women and of couples.

Currently the way of dealing with ovarian failure and other causes of infertility is through assisted reproductive technologies are procedures that involve extracorporeal handling of both human eggs (oocytes or ova) and sperm (spermatozoa), and of embryos for the purpose of establishing a pregnancy in a female subject. These procedures include, but are not limited to, in vitro fertilization (“IVF”) including embryo transfer, gamete intrafallopian transfer, zygote intrafallopian transfer, tubal embryo transfer, gamete and embryo cryopreservation, oocyte and embryo donation, and gestational surrogacy. In vitro fertilization (“IVF”) has evolved as the major treatment for infertility or sub-fertility when other methods of assisted reproductive technology have failed. In its most basic sense, the process involves extracting the female egg from a woman and fertilizing the egg by sperm outside the body (“in vitro”). The process involves monitoring a woman's ovulatory process, removing multiple eggs from the woman's ovaries and letting sperm fertilize the eggs in a fluid medium in a laboratory. The eggs are usually retrieved from the patient by transvaginal oocyte retrieval involving an ultrasound-guided needle piercing the vaginal wall to reach the ovaries. Through this needle, follicles can be aspirated, and the follicular fluid is handed to the IVF laboratory to identify and diagnose the ova. It is common to remove between ten and thirty eggs from each patient. The fertilized egg, (embryo), or usually multiple embryos, are then transferred to the patient's uterus with the intention of establishing a successful pregnancy. See, for example, U.S. Pat. No. 7,781,207. Unfortunately, the timing of implantation is sometimes skewed due to inefficiencies of the endometrium to respond to hormonal signaling. In this patent, we describe means of healing the endometrium in order to increase endometrial activity and allow for proper implantation and development of the embryo.

### SUMMARY

Preferred embodiments are directed to methods of preventing and/or treating endometrial atrophy, comprising the steps of: a) selecting a patient in need of treatment; b) administering an effective amount of a gene therapeutic to the patient need of treatment; c) assessing effect of said gene therapeutic infusion and adjusting concentration and frequency based on response.

Preferred methods include embodiments wherein said gene therapeutic is administered into the ovarian artery.

Preferred methods include embodiments wherein said gene therapeutic is administered in the form of free DNA plasma.

Preferred methods include embodiments wherein said gene therapeutic is administered in the form of free mRNA.

Preferred methods include embodiments wherein said gene therapeutic is administered in the form of an adenoviral vector.

Preferred methods include embodiments wherein said gene therapeutic is administered in the form of a lentiviral vector.

Preferred methods include embodiments wherein said gene therapeutic is administered in the form of a cellular therapy.

Preferred methods include embodiments wherein said gene therapeutic is administered in the form of an exosomal therapy.

Preferred methods include embodiments wherein said gene therapeutic is angiogenic.

Preferred methods include embodiments wherein said angiogenic gene is VEGF.

Preferred methods include embodiments wherein said angiogenic gene is VEGF-C.

Preferred methods include embodiments wherein said angiogenic gene is FGF-1.

Preferred methods include embodiments wherein said angiogenic gene is FGF-2.

Preferred methods include embodiments wherein said angiogenic gene is FGF-5.

Preferred methods include embodiments wherein said angiogenic gene is FGF-22.

Preferred methods include embodiments wherein said angiogenic gene is EGF-1.

Preferred methods include embodiments wherein said angiogenic gene is PDGF-BB.

Preferred methods include embodiments wherein said angiogenic gene is angiopoietin.

Preferred methods include embodiments wherein said angiogenic gene is interleukin-20.

Preferred methods include embodiments wherein said angiogenic gene is HGF-1.

Preferred methods include embodiments wherein said angiogenic gene is G-CSF.

Preferred methods include embodiments wherein said angiogenic gene is SDF-1.

Preferred methods include embodiments wherein said angiogenic gene is NGF-1.

Preferred methods include embodiments wherein said angiogenic gene is BDNF.

Preferred methods include embodiments wherein said angiogenic gene is CNTF.

Preferred methods include embodiments wherein said gene therapeutic is anti-apoptotic.

Preferred methods include embodiments wherein said anti-apoptotic gene is soluble fas ligand.

Preferred methods include embodiments wherein said anti-apoptotic gene is bcl-2.

Preferred methods include embodiments wherein said anti-apoptotic gene is bcl-2xL.

Preferred methods include embodiments wherein said anti-apoptotic gene is livin.

Preferred methods include embodiments wherein said anti-apoptotic gene is survivin.

Preferred methods include embodiments wherein said anti-apoptotic gene is IAP-1.

Preferred methods include embodiments wherein said anti-apoptotic gene is IAP-2.

Preferred methods include embodiments wherein said anti-apoptotic gene is interleukin-1.

Preferred methods include embodiments wherein said anti-apoptotic gene is interleukin-3.

Preferred methods include embodiments wherein said anti-apoptotic gene is interleukin-10.

Preferred methods include embodiments wherein said anti-apoptotic gene is nf-kappa p55.

Preferred methods include embodiments wherein said anti-apoptotic gene is nf-kappa p65.

Preferred methods include embodiments wherein said anti-apoptotic gene is Mn-SOD.

Preferred methods include embodiments wherein said anti-apoptotic gene is Mg-SOD.

Preferred methods include embodiments wherein said anti-apoptotic gene is bcl-w.

Preferred methods include embodiments wherein said anti-apoptotic gene is mcl-1.

Preferred methods include embodiments wherein said anti-apoptotic gene is api-5.

Preferred methods include embodiments wherein said anti-apoptotic gene is Bag-1.

Preferred methods include embodiments wherein said anti-apoptotic gene is Bag-2.

Preferred methods include embodiments wherein said anti-apoptotic gene is BCL-2a1.

Preferred methods include embodiments wherein said anti-apoptotic gene is BCL-2l1.

Preferred methods include embodiments wherein said anti-apoptotic gene is BCL-1l1

Preferred methods include embodiments wherein said anti-apoptotic gene is Birc1b.

Preferred methods include embodiments wherein said anti-apoptotic gene is Birc4.

Preferred methods include embodiments wherein said anti-apoptotic gene is BNIP.

Preferred methods include embodiments wherein said anti-apoptotic gene is BNIP-2.

Preferred methods include embodiments wherein said anti-apoptotic gene is BNIP-3.

Preferred methods include embodiments wherein said anti-apoptotic gene is CFLAR.

Preferred methods include embodiments wherein said anti-apoptotic gene is Dad1.

Preferred methods include embodiments wherein said anti-apoptotic gene is Faim.

Preferred methods include embodiments wherein said anti-apoptotic gene is Polb.

Preferred methods include embodiments wherein said anti-apoptotic gene is prdx2.

Preferred methods include embodiments wherein said anti-apoptotic gene is PRLR.

Preferred methods include embodiments wherein said anti-apoptotic gene is TRAF-1.

Preferred methods include embodiments wherein said anti-apoptotic gene is TRAF-3.

Preferred methods include embodiments wherein said anti-apoptotic gene is TRAF-4.

Preferred methods include embodiments wherein said gene therapeutic is anti-fibrotic.

Preferred methods include embodiments wherein said anti-fibrotic gene is antisense to TGF-beta.

Preferred methods include embodiments wherein said anti-fibrotic gene is an inhibitor of SMAD2.

Preferred methods include embodiments wherein said anti-fibrotic gene is an inhibitor of SMAD4.

Preferred methods include embodiments wherein said anti-fibrotic gene is MMP1.

Preferred methods include embodiments wherein said anti-fibrotic gene is an inhibitor of MMP2.

Preferred methods include embodiments wherein said anti-fibrotic gene is an inhibitor of MMP3.

Preferred methods include embodiments wherein said anti-fibrotic gene is an inhibitor of MMP7.

Preferred methods include embodiments wherein said anti-fibrotic gene is an inhibitor of MMP9.

Preferred methods include embodiments wherein said anti-fibrotic gene is an inhibitor of MMP13.

Preferred methods include embodiments wherein said gene therapeutic are directly injected into the uterine lining with or without ultrasound guidance.

Preferred methods include embodiments wherein said gene therapeutic are directly injected into the uterine lining via transvaginal approach.

Preferred methods include embodiments wherein said gene therapeutic are directly injected into the uterine lining via laparoscopic approach.

Preferred methods include embodiments wherein said gene therapeutic are placed in carrier solution of less than 3 percent hematocrit platelet rich plasma for injection.

Preferred methods include embodiments wherein said gene therapeutic are placed in carrier solution of reconstituted lyophilized or fresh platelet lysate for injection.

Preferred methods include embodiments wherein the carrier solution of less than 3 percent hematocrit platelet rich plasma or reconstituted lyophilized or fresh platelet lysate for injection is use for injection to prime the tissue.

### DETAILED DESCRIPTION OF THE INVENTION

The invention provides administration of gene therapy as a means of preventing or treatment of endometrial failure. In some embodiments endometrial failure occurs as a result of natural causes. In other cases, endometrial failure is caused by genetic or artificial causes such as exposure to inhibitors of proliferation. In other cases, endometrial failure is caused by endometrial inflammation and/or fibrosis.

“Asherman's syndrome” (AS) (or Fritsch syndrome) is a condition characterized by adhesions and/or fibrosis of the endometrium most often associated with dilation and curettage of the intrauterine cavity. A number of other terms have been used to describe the condition and related conditions including: intrauterine adhesions (IUA), uterine/cervical atresia, traumatic uterine atrophy, sclerotic endometrium, endometrial sclerosis, and intrauterine synechiae. Trauma to the endometrial basal layer, for example, after a dilation and curettage (D&C) performed after a miscarriage, or delivery, or for medical abortion, can lead to the development of intrauterine scars resulting in adhesions that can obliterate the uterine cavity to varying degrees. In the extreme, the whole cavity can be scarred and occluded. Even with relatively few scars, the endometrium may fail to respond to estrogen, and a subject may experience secondary menstrual irregularities (such as amenorrhea, hypomenorrhea, or oligomenorrhea) and become infertile. AS can also result from other pelvic surgeries including cesarean sections, removal of fibroid tumors (myomectomy) and from other causes such as IUDs, pelvic irradiation, schistosomiasis and genital tuberculosis. Chronic endometritis from genital tuberculosis is a significant cause of severe intrauterine adhesions (IUA) in the developing world, often resulting in total obliteration of the uterine cavity which is difficult to treat.

“Blastocyst” is an embryo, five or six days after fertilization, having an inner cell mass, an outer cell layer called the trophectoderm, and a fluid-filled blastocele cavity containing the inner cell mass from which the whole of the embryo is derived. The trophectoderm is the precursor to the placenta. The blastocyst is surrounded by the zona pellucida which is subsequently shed when the blastocyst “hatches.” The zona pellucida, composed of a glycoprotein coat, surrounds the oocyte from the one-cell stage to the blastocyst stage of development. Prior to embryo attachment and implantation, the zona pellucida is shed from the embryo by a number of mechanisms including proteolytic degradation. The zona pellucida functions initially to prevent entry into the oocyte by more than one sperm, then later to prevent premature adhesion of the embryo before its arrival into the uterus.

“Cryo-IVF” refers to a process of in vitro fertilization in which the embryo is cryopreserved then thawed prior to embryo transfer or a process in which the oocyte used for fertilization has been previously frozen then thawed. “Fresh-IVF” refers to a process of in vitro fertilization wherein the embryo is not frozen prior to transfer into the uterine cavity and where the oocytes used to prepare the embryo have not previously been frozen.

“Embryo” is the product of the division of the zygote to the end of the embryonic stage, eight (8) weeks after fertilization. The cleavage stage of the embryo occurs during the first three days of culture. “Embryo transfer” is the procedure in which one or more embryos and/or blastocysts are placed into the uterus or fallopian tubes. As such, the terms “blastocyst” and “embryo” are, used interchangeably herein for purposes of defining the term “embryo transfer” and in application of the term “embryo transfer” within the scope and application of the invention as described and claimed.

“Endometrium” refers to the tissue lining the internal surface of the uterus, which is composed of a layer of epithelial cells. The embryo first comes into contact with the endometrium and the extracellular matrix (the “mucus”) for implantation. The epithelial and underlying stromal cell layer cyclically thickens, secretes mucus and is shed from the body under the hormonal influences of the menstrual cycle. By the term “implantation” herein is meant the attachment and subsequent penetration by the blastocyst (after having shed its zona pellucida), usually into the endometrium. Attachment to the endometrium lining may occur by interaction between the attachment molecules and on or more components of the endometrium, including membranes of the epithelial cells, mucus, mucin components of the mucus, or an exogenously introduced component of the uterus.

“Fertilization” refers to the penetration of the ovum by the spermatozoa and combination of their genetic material resulting in the formation of a zygote. “Initiation of in vitro fertilization” as used herein means the initiation of controlled ovarian stimulation of a female patient, which involves pharmacological treatment in which women are stimulated to induce the development of multiple ovarian follicles to obtain multiple oocytes at follicular aspiration.

“Uterus”, commonly referred to as the womb, is the major female hormone-responsive reproductive sex organ of most mammals including humans that contains the cervix at one end while the other is connected to one or both fallopian tubes, depending on the species. The reproductive function of the uterus is to accept a fertilized ovum which passes through the utero-tubal junction from the fallopian tube. It implants into the endometrium, and derives nourishment from blood vessels which develop exclusively for this purpose. The fertilized ovum becomes an embryo, attaches to a wall of the uterus, creates a placenta, and develops into a fetus during gestation until childbirth. As used herein, the term “uterus” incorporates the fallopian tubes for purposes of embryo transfer. The term “uterus” is also used interchangeably herein with the term “uterine cavity,” which is the cavity of the body of the uterus.

In the current day treatment of infertility, one of the missing elements is methods of healing endometrium that has been damaged. This current patent teaches that administration of therapeutic genes, such as antioxidant, anti-inflammatory, regenerative, and antifibrotic genes may be used to enhance ability of the endometrium to respond to various factors associated with endometrial failure. Currently there are techniques used routinely in order to increase the chance of pregnancy in infertile woman that can benefit from the use of gene therapy of the endometrium. It is known that the most common is ovarian hyperstimulation or super-ovulation that is used in order to stimulate the ovaries to produce multiple eggs that are then retrieved from the patient. The long protocol typically involves downregulation (suppression or exhaustion) of the pituitary ovarian axis by the prolonged use of a gonadotropin releasing hormone (GnRH) agonist. Subsequent ovarian hyperstimulation, typically using follicle stimulating hormone (FSH), starts once the process of downregulation is complete, generally after 10 to 14 days. An IVF cycle using this protocol is known as conventional in vitro fertilization. The short protocol skips the downregulation procedure, and consists of a regimen of fertility medications to stimulate the development of multiple follicles of the ovaries. Other procedures use gonadotrophin-releasing hormone agonists (GnRHA), which decreases the need for monitoring by preventing premature ovulation, and more recently gonadotrophin-releasing hormone antagonists (GnRH Ant) have been used, which have a similar function. In most patients, injectable gonadotropins (usually FSH analogues) are used under close monitoring. Such monitoring frequently checks the estradiol level of the patient and, by means of gynecologic ultrasonography, follicular growth. Typically, approximately 10 days of injections are necessary. Ovarian stimulation carries the risk of excessive stimulation leading to ovarian hyperstimulation syndrome (OHSS), a potentially life-threatening complication of abdominal distension, ovarian enlargement, respiratory, hemodynamic and metabolic complications. In addition, it has also of recent been demonstrated that fertility drugs used for the stimulation of ovulation of patients undergoing IVF treatment contribute to compromised implantation receptivity of the embryo in the uterus and lead to a decreased rate of pregnancy inception. Therefore, the invention aims to increase the possibility of successful pregnancy by repairing the endometrium.

In one of the embodiments of the invention, provided is a method of enhancing success of in vitro fertilization for a female patient, the method comprising the steps of introducing into the uterus an effective amount of a composition comprising gene therapy in the form of either plasmid, mRNA, circular DNA, viral based deliver and quantum dot based delivery. Said gene therapy is utilized to “condition” or “repair” the endometrium prior to transferring at least one embryo into the uterus of the patient after a predetermined delay following initiation of the in vitro fertilization of said patient; and wherein the method results in an increase in the probability of implantation of the embryo in the uterus with successful inception of pregnancy when compared to in vitro fertilization methods lacking such steps. The predetermined delay of time is a period of time sufficient to decrease autoimmune rejection of the embryo or the risk of autoimmune response of the patient. The preferred delay of time before embryo transfer is at least two menstrual cycles or two cycles of ovulation of the patient, though the delay will vary from patient to patient and among species, hybrids and varieties of animals. The preferred delay of time in a human patient is three to twelve months. It is another aspect of the invention that toward the end of the time-delay period, the women's endometrium is prepared by the use of gene therapy in a way so as to optimize the acceptance of the embryo by the uterine cavity. According to one embodiment of the invention, this is accomplished by intrauterine injection of peripheral blood mononuclear cells (PBMCs), most preferably obtained from the patient that have been transfected with the gene of interest. More particularly, disclosed is a method of in vitro fertilization for a female patient involving the steps of: (a) obtaining at least one oocyte and fertilizing the oocyte with spermatozoa to form a zygote; (b) developing the zygote in vitro to an embryo stage; ((c) cryopreserving the embryo; (d) waiting a predetermined period of time sufficient to decrease the risk of autoimmune response of the patient; (e) extracting a first portion of peripheral blood mononuclear cells (PBMCs) from the blood of the patient 2 to 4 days prior end of waiting period; (f) culturing said first portion of PBMCs in a suitable culture medium c (g) extracting a second fresh portion of PBMCs from the blood of the patient on the last day of the waiting period; (h) combining the cultured first portion of PBMCs with the fresh second portion of PBMCs to obtain a composition comprising fresh and cultured PBMCs; (i) introducing the composition of PBMCs into the uterus of the patient; (j) thawing the embryo from the cryopreserved state; and (k) transferring at least one thawed embryo into the uterus of the patient to effectuate pregnancy. An additional aspect of the invention is a composition comprising PBMCs, a method for producing the composition and the application of the PBMC composition in IVF treatment and for growing and engineering certain target tissue of an organism, most preferably the endometrium of a female uterus. Such method involves extracting PBMCs from the blood of a patient; propagating a portion of the extracted PBMCs in the presence of 4.8-6.0% carbon dioxide (CO2) at 36.7-37.3 degrees C. in a culture medium containing (i) RPMI 1640 medium with L-glutamine and sodium bicarbonate, (ii) human recombinant albumin and (iii) a promoting agent capable of improving the ability of PBMCs to enhance tissue growth, such as human chorionic gonadotropin (hCG); and combining the fresh and cultured portions of PBMCs to obtain said composition.

As used herein, a peripheral blood mononuclear cell (PBMC) is a multipotent cell that is extracted, harvested, derived, isolated or otherwise obtained from the blood of a subject. A PBMC is a blood cell and that has a round nucleus. The PBMC class includes, but is not limited to lymphocytes, a monocytes and macrophages. These blood cells are a critical component in the immune systems of organisms utilized in fighting infection and in operating other functions involving the immune system. The lymphocyte population consists of T cells (CD4 and CD8+ about 75%), B cells and NK cells (about 25% combined). The PBMC population also includes basophils and dendritic cells.

PBMCs can be isolated from human peripheral blood by common methods known in the art. The cells can be extracted from whole blood using FICOLL® (GE Healthcare Bio-Sciences AB LLC of Sweden), a hydrophilic polysaccharide that separates layers of blood, which will separate the blood into a top layer of plasma, followed by a layer of PBMCs and a bottom fraction of leukocytes, erythrocytes, and polymorphonuclear cells (such as neutrophils, eosinophils). The polymorphonuclear cells can be further isolated by lysing the red blood cells. Ficoll® is part of Ficoll-Paque® (GE Healthcare Bio-Sciences AB LLC of Sweden). Ficoll-Paque® is normally placed at the bottom of a conical tube, and blood is then slowly layered above the Ficoll-Paque®. After being centrifuged, several layers will be visible in the conical tube, from top to bottom: plasma and other constituents, the layer of mononuclear cells containing the PBMCs, Ficoll-Paque®, and erythrocytes and granulocytes which are present in pellet form. This separation allows easy harvest of the PBMC's. Some red blood cell trapping (presence of erythrocytes and granulocytes) may occur in the PBMC or Ficoll-Paque® layer. Major blood clotting may sometimes occur in the PBMC layer. Ethylene diamine tetra-acetate (EDTA) and heparin are commonly used in conjunction with Ficoll-Paque® to prevent clotting. Because Ficoll-Paque® layering is a very slow process, devices that aid in the overlay, which is most time-consuming step, have been developed. One such a product is SepMate™-50 (StemCell Technology Inc. of Canada), a specialized tube containing a porous insert that forms a physical barrier between the Ficoll-Paque® and blood sample. This allows the blood sample to be rapidly pipetted onto the insert, avoiding the need for overlaying it directly onto Ficoll-Paque®. The SepMate™ insert also reduces the duration of the centrifugation step, and after centrifugation, the top layer containing plasma and PBMCs can be poured into a separate container. Other devices include a column containing a porous high-density polyethylene barrier or “fit.” These products allow blood to be layered on much more quickly without mixing polysaccharide and blood. An example of such a product is the “Accuspin System Histopaque-1077” sold by Sigma Aldrich. It is also possible to have the Ficoll-Paque® separating system included in a vacutainer blood collection tube. Such vacutainers increase the convenience and safety of collecting blood products, but are much more costly than the basic vacutainer. Another such product, Floaties™, has been shown to effectively overlay blood or a cellular suspension on Ficoll® using a special mixture of polymer beads or pellets. This product is inexpensive, reduces researcher reliance on technique, and actually speeds up the overlay process. Any of the foregoing techniques, including other techniques that are or will become practiced in art of collecting, isolating, extracting, harvesting, separating, removing, or in any other way obtaining PBMCs from the blood of an organism, human or animal, are contemplated within the scope of the embodiments of the invention herein.

In one of the aspects of the invention, provided herein is a method of culturing the PBMCs cells. The method comprises culturing the cells on fibronectin-coated plates in a humidified atmosphere containing from 4.8% to 6.0% of carbon dioxide (CO2) at a temperature in the range of 36.7.degree. C. to 37.3.degree. C., at a density of 104 to 107/mL. In a preferred embodiment of the method, PBMCs are cultured for a period of time in the range of 46 hours to 72 hours. In a most preferred embodiment, the PBMCs are cultured on fibronectin-coated plates in an atmosphere of 5.0% of CO2 at a temperature of 37.1 degree C. for 48 hours.

The culture media used herein for propagating the extracted PBMCs is a Roswell Park Memorial Institute medium, commonly known as RPMI medium, available from a number of sources. RPMI medium is often used for cell and tissue culture. RPMI 1640 medium has traditionally been used for the growth of serum-free human lymphoid cells, bone marrow cells and hybridoma cells. RPMI 1640 medium uses a bicarbonate buffering system and differs from most mammalian cell culture media in its pH 8 formulation. The preferred medium according to the methods of the invention utilizes RPMI 1640 culture medium containing L-glutamine and sodium bicarbonate.

According to another aspect of the invention, human recombinant albumin (HRA) is added to the culture medium. HRA is a well-known carrier protein present in high concentrations in plasma with a circulatory half-life of approximately 19 days. It functions in fatty acid transportations to tissues, protein stabilization, binding metal ions to surfaces, and an antioxidative effect in plasma. HRA is widely available from a number of providers, for example from Novozymes, Inc. or Sigma-Aldrich, LLC. The addition of HRA during the method of the invention to the culture medium acts as a food supplement that improves the growth of the PBMCs.

In some embodiments of the invention, MSC are utilized as the cellular vector. In such an embodiment, MSC are transfected with therapeutic genes. In one embodiment of the invention, Wharton's jelly mesenchymal stem cells are used as a source of immune modulatory cells. Wharton's jelly cells are derived from umbilical cords that are obtained from healthy mothers that have no history of genetic diseases or cancer, and have been tested negative for hepatitis B/C virus, human immunodeficiency virus, Epstein-Barr virus, cytomegalovirus and syphilis in serum. Manufacturing of Wharton's jelly mesenchymal stem cells is performed in under sterile conditions, for example in a laminar flow hood. During the process of manufacturing, it is ideal for the production to occur in a class 10,000 clean production suite. Each technician properly gowns when entering in the GMP room. Before entry into the clean lab area, the technician obtains a bunny suit in the ante room. After the hood of the bunny suit is placed on, a mouth covering is put on, making sure that all hair is fully covered under the hood and mouth covering. The technician puts on a pair of sterile powder free gloves, and enters the clean lab space with the sample. Environmental monitoring is performed in the Class 10,000 clean room. The umbilical cord is washed with phosphate buffered saline (PBS) twice and then dissected with scissors into pieces approximately one cubic centimeter in volume. The tissue is subsequently plated into a culture dish in low-DMEM medium supplemented with 5-10% platelet rich plasma or fetal calf serum. Cell cultures are maintained in a humidified atmosphere with 5% CO2 at 37° C. After approximately 3 days of culture, the medium is replaced to remove the tissue and non-adherent cells, and the media is changed twice weekly thereafter. Once 80% confluence is reached, the adherent cells (passage 0) were detached with approximately 0.125% trypsin and passaged in the cell culture dish. The Wharton's jelly mesenchymal stem cells are cultured and expanded for 4-6 passages to prepare final cell products. The cellular product is assessed for contamination, including aerobic and anaerobic bacteria, mycoplasma, HBV, HCV, HIV, EBV, CMV, syphilis, and endotoxin testing. To assess purity, cells must possess >90% expression of CD90 and CD105 and <5% CD34, CD45 and HLA-DR. Additionally, cells must have a chromosomal karyotype of UC-MSC was normal.

For production of mesenchymal stem cells, reagent qualification may be necessary. The qualification process begins with the vender of the reagent. The vender is qualified through our standard operating procedure. A corresponding form is completed and approval gained before a vender can be used. The criteria identified as important in qualifying a supplier include quality of product, services offered, competitive pricing, communication, availability, how complaints are handled and the overall fit to our systems. This list is not all inclusive. Quality Systems reviews each qualification form and will approve based on the criteria stated above. Once the vender is approved, they are added to the Supplies and Services List. Associates ordering supplies including reagents use the list. Only approved venders on the list are used by associates ordering supplies involving reagents. Once the reagent arrives, it is logged on the Supplies Receipt, Inspection and Inventory Log. The form instructs the associate to complete certain information for the incoming reagent. These fields are date received, initials of receiver, name of the item, manufacturer, lot number, expiration date, package passed visual inspection, product passed visual inspection, date available for use and quantity. The COA is examined for reagents and placed in the applicable COA binder under that reagent name. These binders are retained per the record retention procedure. Once this is completed the reagent is released from quarantine and placed in the applicable area. If the reagent needs refrigerated or is to remain frozen, it is placed in the applicable storage environment. FDA or other national regulatory body-approved reagents are used if available. In one embodiment, an excipient used in the cryopreservation of the cells is Dimethyl Sulfoxide (DMSO). Each dose of mesenchymal stem cell may be cryopreserved using 10% DMSO, or 2 mL of DMSO in a total volume of 10 mL of final product. Infusion of this amount of DMSO is well within the safety parameters for a 30 kg child; Pediatric Stem Cell Transplant SOP states that the maximum dose of DMSO is 15 mg/kg/dose. For intralymphatic, or perilymphatic administration, various amounts of cells may be used, as well as numerous lymphatic locations.

In addition to mesenchymal stem cells, the invention may be practiced by administration of Sertoli cells via perilymphatic, or intralymphatic administration. One of skill in the art is directed towards means and methods of isolating Sertoli cells within the scope of the invention, include, patent documents, WO 95/28167, WO 96/28174, WO 98/28030, WO 00/27409, WO 2000/035371, WO 2005/018540, US Pat. App. Pub. 2005/0118145 and U.S. Pat. Nos. 5,725,854, 5,843,340, 5,849,285, 5,948,422, 5,958,404, 6,149,907, 6,303,355, 6,649,160, 6,716,246, 6,783,964, 6,790,441, and 6,958,158.

In some embodiments, the Sertoli cells used for the practice of the invention are adult Sertoli cells. The term “adult”, as used herein, refers to age of a sexually mature male from which the cells are extracted. For this disclosure, sexual maturity is the developmental stage at which a being can reproduce, for example, male rats reach sexual maturity at 3 months, male mice reach sexual maturity at 5-7 weeks and male pigs reach sexual maturity at 6-9 months of age. In illustrative embodiments, the Sertoli cells are porcine cells derived from about 1 to 2-year-old boars. Alternatively, the Sertoli cells of the invention may be obtained from any suitable source, for example, cows, horses, dogs, cats, rabbits, primates (human or non-human (e.g., monkeys, chimpanzees)), etc. In other embodiments, Sertoli cells may be derived from a neonatal or fetal animal. Furthermore, Sertoli cells may be generated from stem cells, such as from bone marrow, embryonic stem cells, inducible pluripotent stem cells, or somatic cell nuclear transfer generated stem cells. In some embodiments, the Sertoli cells of the invention have been selected based on expression of immune suppressive molecules, for example Fas ligand. The isolated Sertoli cells may and often do contain other cell types naturally present in the testes, including endothelial cells, Leydig cells, etc. Accordingly, pharmaceutical compositions of the invention may further comprise non-Sertoli cells, including cells that are naturally present in the testes and are, therefore, co-isolated with Sertoli cells. Furthermore, the Sertoli cells of the invention may be primary cells or cell lines derived from such primary cells.

Sertoli cells of the invention may be genetically altered, for example, they may be genetically modified to express, and optionally, secrete one or more immune modulatory factors. Examples of such factors include BLC, Eotaxin-1, Eotaxin-2, G-CSF, GM-CSF, I-309, ICAM-1, IL-1 ra, IL-2, IL-4, IL-5, IL-6 sR, IL-7, IL-10, IL-13, IL-16, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Ax1, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 RI, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-1ACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK. Methods for cell transfection and transformation are known to one of skill in the art. Methods of gene therapy by transfection of genes into Sertoli cells are described, for example, in Dufour et al., Cell Transplant. (2004) 13(1):1-6 and Trivedi et al., Exp. Neurol. (2006) 198, 88-100.

Additionally, pharmaceutical compositions of the invention may comprise non-testicular cells. For example, Sertoli cells may be co-cultured and/or transplanted with another cell type, which benefits from the immunoprotective effect of the Sertoli cells. Specific examples of such other cell types include those that either naturally produce or were modified to produce immune modulatory factors, such as those listed above. In some embodiments Sertoli cells are administered with autoantigens for the purpose of inducing antigen-specific tolerance.

In the case of placental tissue, which represents an almost unlimited supply of MSC, placenta is collected from delivery procedures, the tissue may be placed in sterile containers with phosphate buffered saline (“PBS”), penicillin/streptomycin and amphotericin B during collection. This may be performed when collecting testicular or ovarian tissue as well. Specifically, harvested tissue is first surface sterilized by multiple washes with sterile PBS, followed by immersion in 1% povidoneiodine (“PVP-1”) for approximately 2 minutes, immersion in 0.1% sodium thiosulfate in PBS for approximately 1 minute, and another wash in sterile PBS. Next the tissue is dissected into 5 g pieces for digestion. Enzymatic digestion is performed using a mixture of collagenase type I and type II along with thermolysin as a neutral protease. The digestion occurs in a 50 cc sterile chamber for 20-45 minutes until the tissue is disaggregated and the suspending solution is turbid with cells. Next the solution is extracted leaving behind the matrix, and cold (4 degree C.) balanced salt solution with fetal bovine serum at 5% concentration is added to quench the enzymes. This resulting suspension is centrifuged at 600.times.g, supernatant is aspirated and MESENCULT® complete medium (basal medium containing MSC stimulatory supplements available from StemCell Technologies, Vancouver, British Columbia) is added to a final volume of approximately 1.5 times the digestion volume to neutralize the digestion enzymes. This mixture is centrifuged at 500 g for 5 minutes, and the supernatant aspirated. The cell pellet is then re-suspended in fresh 10 MESENCULT® complete medium plus 0.25 mg/mL amphotericin B, 100 IU/mL penicillin-G, and 100 mg/mL streptomycin (JR Scientific, Woodland, Calif.).

Cells are then plated at an initial concentration of approximately one starting 5 g tissue digest per 225 cm2 flask. Culture flasks are monitored daily and any contaminated flasks removed immediately and recorded. Non-contaminated flasks are monitored for cell growth, with medium changes taking place three times per week. After 14 days of growth, MSC are detached using 0.25% trypsin/1 mM EDTA (available from Invitrogen, Carlsbad, Calif.). Cell counts and viability were assessed using flow cytometry techniques and cells are banked by controlled rate freezing in sealed vials. For the preparation of bone marrow MSC, bone marrow is collected and placed within a “washing tube”. Before the collection procedure a “washing tube” is prepared in the class 100 Biological Safety Cabinet in a Class 10,000 GMP Clean Room. To prepare the washing tube, 0.2 mL amphotericin B (Sigma-Aldrich, St Louis, Mo.), 0.2 mL penicillin/streptomycin (Sigma 50 ug/nl) and 0.1 mL EDTANa2 (Sigma) is added to a 50 mL conical tube (Nunc) containing 40 mL of GMP-grade phosphate buffered saline (PBS). Specifically, the washing tube containing the collected bone marrow is topped up to 50 mL with PBS in a class 100 Biological Safety Cabinet and cells are washed by centrifugation at 500 g for 10 minutes at room temperature, which produced a cell pellet at the bottom of the conical tube. Under sterile conditions supernatant is decanted and the cell pellet is gently dissociated by tapping until the pellet appeared liquid. The pellet is re-suspended in 25 mL of PBS and gently mixed so as to produce a uniform mixture of cells in 30 PBS. In order to purify mononuclear cells, 15 mL of Ficoll-Paque (Fisher Scientific, Portsmouth N.H.) density gradient was added underneath the cell-PBS mixture using a 15 mL pipette. The mixture is subsequently centrifuged for 20 minutes at 900 g. Thereafter, the buffy coat is collected and placed into another 50 mL conical tube together with 40 mL of PBS. Cells are then centrifuged at 400 g for 10 minutes, after which the supernatant is decanted and the cell pellet re-suspended in 40 mL of PBS and centrifuged again for 10 minutes at 400 g. The cell pellet is subsequently re-suspended in 5 mL complete DMEM-low glucose media (GibcoBRL, Grand Island, N.Y.) supplemented with approximately 20% Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot is used for all experiments. Additionally, the media is supplemented with 1% penicillin/streptomycin, 1% amphotericin B, and 1% glutamine. The re-suspended cells are mononuclear cells substantially free of erythrocytes and polymorphonuclear leukocytes as assessed by visual morphology microscopically. Viability of the cells was assessed with trypan blue. Only samples with >90% viability were selected for cryopreservation in sealed vials. For preparation of MSC from teeth, said teeth are extracted under sterile conditions and placed into sterile chilled vials containing 20 mL of phosphate buffered saline with penicillin/streptomycin and amphotericin B (Sigma-Aldrich, St. Louis, Mo.). Teeth were thereafter externally sterilized and processed first 20 by washing several times in sterile PBS, followed by immersion in 1% povidoneiodine (PVP-1) for 2 minutes, immersion in 0.1% sodium thiosulfate in PBS for 1 minute, followed by another wash in sterile PBS. The roots of cleaned teeth is separated from the crown using pliers and forceps to reveal the dental pulp, and the pulp is placed into an enzymatic bath consisting of type I and type II collagenase (Vitacyte, Indianapolis, USA) with thermolysin as the neutral protease. Pulp tissue is allowed to incubate at 37 degree C. for 20-40 min to digest the tissue and liberate the cells. Once digestion is complete, MESENCULT® complete medium is added to a final volume of 1.5 times. the digestion volume to neutralize the digestion enzymes. This mixture is centrifuged at 500 g for 5 min, and the supernatant aspirated. The cell pellets are resuspended in fresh MESENCULT® complete medium plus 0.25 mg/mL amphotericin B, 100 30 IU/mL penicillin-G, and 100 mg/mL streptomycin (JR Scientific, Woodland, Calif.). Cells are plated at an initial concentration of one tooth digest per 25 cm.sup.2 flask. Culture flasks are monitored daily and any contaminated flasks removed immediately and recorded. Non-contaminated flasks were monitored for cell growth, with medium changes taking place three times per week. After 14 days of growth, MSC are detached using 0.25% trypsin/1 mM EDTA (Invitrogen, Carlsbad, Calif.), cell counts and viability were assessed using a standard trypan blue dye exclusion assay (Sigma) and hemacytometer, and bAU3 the DPSC divided equally between two 75 cm.sup.2 flasks. After the first passage, DPSC cultures were harvested once they reach 7080% confluence. These cells are then cryopreserved in sealed vials. MSCs from the skin, including epidermal, dermal, and subcutaneous tissue of healthy adult patients undergoing cosmetic plastic surgery are isolated by collagenase digestion procedure. Once received, the tissue is cleaned of any unwanted adipose tissue and hair. The tissue is then sterilized using 1 times PVP-iodine solution and 1 times sodium thiosulfate followed by washing twice in sterile PBS. The dermis is then minced into 1 mm.sup.3 pieces following collagenase enzymatic digestion for 30-40 minutes at 37 degrees C. Afterwards, tissue pieces were dissociated by pipetting into 5 mL pipette and centrifuged at 300 g for 5 min The pellet was suspended in cell growth media Dulbecco's Modified Eagle Medium: Nutrient Mixture F-12 (“DMEM/F12”) (available from Invitrogen, Carlsbad, Calif.) (1:1) containing amphoterecin, penicillin and streptomycin supplemented with 10% fetal bovine serum. Cell suspensions were transferred into T-tissue culture flask and grown until 80-90% confluence. The cells were placed in a T-75 flask before being used for flow analysis and differentiation. Another embodiment of the invention is the use of MSCs from the umbilical cord during harvested during delivery. Once received, the tissue i washed two to three times in sterile PBS and then divided into pieces of approximately 5 grams each. Thereafter, the tissue is decontaminated, and each 5 gram aliquot of tissue is placed in a sterile 100 mm tissue culture dish, and covered with a lid to prevent drying. The tissue was dissociated via enzymatic digestion in 50 cc tubes, and is minced into fragments less than 1 mm.sup.3 using a sterile scalpel. Then, the chopped tissue is placed in an enzyme bath, and the tube is capped and transferred to an incubator. The tubes were swirled for fifteen seconds every ten minutes for forty minutes. Thereafter, the digesting enzyme was diluted by adding 45 mL of cold DME/F12 complete media (FBS, Pen/Strep and Amphotericin B), with the tubes being capped and inverted to mix the contents. Next, the tubes were centrifuged at 400.times.g for fifteen minutes on low break. The top media is aspirated using a 25 mL pipette by leaving approximately 5 mL at the bottom of the tube, with special care being taken to aspirate the entire medium in the tube. The bottom 5 mL medium (containing tissue fragments and cells including MSCs) was resuspended in fresh 20 mL DME-F12 complete medium mixed well and placed into a t-75 flask, and transferred to an incubator. The tissue is washed off during the first media 10 change after 48 hours post-digestion, and the media was changed three times per week. Cells are grown to 70%-80% confluence and then either passaged, frozen down as passage zero cells, or differentiated. Cells were not allowed to reach confluence or to remain at confluence for extended periods of time.

Cell expansion for cells originating from any of the abovementioned tissues above takes place in clean room facilities purpose built for cell therapy manufacture and meeting GMP clean room classification. In a sterile class II biologic safety cabinet located in a class 10,000 clean production suite, cells were thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot was used for all experiments. Cells are subsequently placed in a T-225 flask containing 45 mL of cDMEM and cultured for 24 hours at 37 degree C. at 5% CO2 in a fully humidified atmosphere. This allowed the MSC to adhere. Non-adherent cells were washed off using cDMEM by gentle rinsing of the flask. This resulted in approximately 6 million cells per initiating T-225 flask. The cells of the first flask were then split into 4 flasks. Cells were grown for 4 days after which approximately 6 million cells per flask were present (24 million cells total). This scheme was repeated but cells were not expanded beyond 10 passages, and were then banked in 6 million cell aliquots in sealed vials for delivery. All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. Donor cells are collected in sterile conditions, shipped to a contract manufacturing facility, assessed for lack of contamination and expanded. The expanded cells are stored in cryovials of approximately 6 million cells/vial, with approximately 100 vials per donor. At each step of the expansion quality control procedures were in place to ensure lack of contamination or abnormal cell growth.

Without departing from the spirit of the invention, mesenchymal stem cells may be optimized to possess heightened immune modulatory properties. In one embodiment this may be performed by exposure of mesenchymal stem cells to hypoxic conditions, specifically hypoxic conditions can comprise an oxygen level of lower than 10%. In some embodiments, hypoxic conditions comprise up to about 7% oxygen. For example, hypoxic conditions can comprise up to about 7%, up to about 6%, up to about 5%, up to about 4%, up to about 3%, up to about 2%, or up to about 1% oxygen. As another example, hypoxic conditions can comprise up to 7%, up to 6%, up to 5%, up to 4%, up to 3%, up to 2%, or up to 1% oxygen. In some embodiments, hypoxic conditions comprise about 1% oxygen up to about 7% oxygen. For example, hypoxic conditions can comprise about 1% oxygen up to about 7% oxygen; about 2% oxygen up to about 7% oxygen; about 3% oxygen up to about 7% oxygen; about 4% oxygen up to about 7% oxygen; about 5% oxygen up to about 7% oxygen; or about 6% oxygen up to about 7% oxygen. As another example, hypoxic conditions can comprise 1% oxygen up to 7% oxygen; 2% oxygen up to 7% oxygen; 3% oxygen up to 7% oxygen; 4% oxygen up to 7% oxygen; 5% oxygen up to 7% oxygen; or 6% oxygen up to 7% oxygen. As another example, hypoxic conditions can comprise about 1% oxygen up to about 7% oxygen; about 1% oxygen up to about 6% oxygen; about 1% oxygen up to about 5% oxygen; about 1% oxygen up to about 4% oxygen; about 1% oxygen up to about 3% oxygen; or about 1% oxygen up to about 2% oxygen. As another example, hypoxic conditions can comprise 1% oxygen up to 7% oxygen; 1% oxygen up to 6% oxygen; 1% oxygen up to 5% oxygen; 1% oxygen up to 4% oxygen; 1% oxygen up to 3% oxygen; or 1% oxygen up to 2% oxygen. As another example, hypoxic conditions can comprise about 1% oxygen up to about 7% oxygen; about 2% oxygen up to about 6% oxygen; or about 3% oxygen up to about 5% oxygen. As another example, hypoxic conditions can comprise 1% oxygen up to 7% oxygen; 2% oxygen up to 6% oxygen; or 3% oxygen up to 5% oxygen. In some embodiments, hypoxic conditions can comprise no more than about 2% oxygen. For example, hypoxic conditions can comprise no more than 2% oxygen.

Enhancement of immune modulatory activity of mesenchymal stem cells may be performed by altering the oxidative stress levels of the patient before, and/or during, and/or after administration of the cells. In one embodiment the patient is treated using mesenchymal stem cells administered intralymphatically or perilymphatically in combination with enhancing the anti-oxidant status of the patient. Enhancement of antioxidant status may be performed through administration of an antioxidant, or combination of antioxidants, said antioxidant may be selected from a group comprising of: ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, intravenous ascorbic acid, polyphenols, pycnogenol, retinoic acid, ACE Inhibitory Dipeptide Met-Tyr, recombinant superoxide dismutase, xenogenic superoxide dismutase, and superoxide dismutase.

In some aspects of the invention, a chemoattractant agent or combination of agents are administered either proximally, or directly to the endometrium being affected by autoimmunity with the purpose of proximally concentrating mesenchymal stem cells to area of inflammation/autoimmunity. Said chemoattractant may be administered in the form of a depot, said depot capable of substantially localizing said chemoattractant is may be selected from a group comprising of: fibrin glue, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol. Furthermore, said chemoattractants useful for the practice of the current invention may be is selected from a group comprising: SDF-1, VEGF, RANTES, ENA-78, platelet derived factors, various isoforms thereof and small molecule agonists of VEGFR-1, VEGFR2, and CXCR4. In another aspect of the invention, the chemoattractant is administered into the area in need, through transfection of a single or plurality of nucleotide(s) encoding said chemoattractant factor. In some embodiments, a perilymphatic or intralymphatic administration of a chemoattractant factor is administered in order to augment retention of mesenchymal stem cells in the lymphatic area.

In another embodiment, mesenchymal stem cells may be optimized for enhanced trafficking and/or immune modulatory activity by genetic modification. Mesenchymal stem cells that expresses or up-regulates expression of a polypeptide, such as, for example, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors .alpha..sub.1.beta..sub.1 and .alpha..sub.2.beta..sub.1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor.alpha.5.beta.1, Factor X, HB-EGF, HBNF, HGF, HUAF, heart derived inhibitor of vascular cell proliferation, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), and/or nicotinic amide. Additionally, mesenchymal stem cells may be transfected with a nucleic acid sequence that induces RNA interference to silence genes associated with pathological immunity such as ABCF1, BCL6, C3, C4A, CEBPB, CRP, ICEBERG, IL1R1, IL1RN, IL8RB, LTB4R, TOLLIP, IFNA2, IL10RA, IL10RB, IL13, IL13RA1, IL5RA, IL9, IL9R, CD40LG (TNFSF5), IFNA2, IL17C, IL1A, IL1B, IL1F10, IL1F5, IL1F6, IL1F7, IL1F8, IL1F9, IL22, IL5, IL-6, IL8, IL9, IL-18, IL-33, LTA, LTB, MIF, SCYE1, SPP1, TNF, CCL13 (mcp-4), CCR1, CCR2, CCR3, CCR4, CCR5, CCR6, CCR7, CCR8, CCR9, CX3CR1, IL8RA, XCR1 (CCXCR1), C5, CCL1 (I-309), CCL11 (eotaxin), CCL13 (mcp-4), CCL15 (MIP-1d), CCL16 (HCC-4), CCL17 (TARC), CCL18 (PARC), CCL19, CCL2 (mcp-1), CCL20 (MIP-3a), CCL21 (MIP-2), CCL23 (MPIF-1), CCL24 (MPIF-2/eotaxin-2), CCL25 (TECK), CCL26, CCL3 (MIP-1a), CCL4 (MIP-1b), CCL5 (RANTES), CCL7 (mcp-3), CCL8 (mcp-2), CXCL1, CXCL10 (IP-10), CXCL11 (I-TAC/IP-9), CXCL12 (SDF1), CXCL13, CXCL14, CXCL2, CXCL3, CXCL5 (ENA-78/LIX), CXCL6 (GCP-2), CXCL9, IL13, and IL8. In some embodiments, mesenchymal stem cells are endowed with augmented antiapoptotic activity by transfection nucleic acids that induce gene silencing to suppress expression of genes associated with induction of apoptosis, such genes include CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, and TRAF4. In another embodiment, mesenchymal stem cells are modulated, either by transfection or other means to enhance expression of anti-apoptotic proteins, such proteins include obestatin, XIAP, survivin, BCL-2, BCL-XL, GATA-4, IGF-1, EGF, heme-oxygenase-1, NF-kB, akt, pi3-k, and epha-2.

The use of mesenchymal stem cells for stimulation of the tolerogenic process may also be performed as an adjuvant to other processes, methodologies, or agents that promote immunological tolerance. Within the definition of immunological tolerance includes suppression of an ongoing autoimmune response, promotion of T regulatory cells, B regulatory cells, tolerogenic dendritic cells, NKT2 cells and type 2 macrophages.

One particular embodiment of the invention is the utilization of mesenchymal stem cells as a means of modulating dendritic cell (DC) function in vitro or in vivo. In one embodiment of the invention, gene therapy is utilized to inhibit complement activation in the endometrium and/or to suppress inflammation. The blood borne protein family of “Complement” was first discovered in the 1890s when it was found to aid or “complement” the killing of bacteria by heat-stable antibodies present in normal serum [1, 2]. The complement system consists of more than 30 proteins that are either present as soluble proteins in the blood or are present as membrane-associated proteins [3]. Activation of complement leads to a sequential cascade of enzymatic reactions, known as complement activation pathways, resulting in the formation of the potent anaphylatoxins C3a and C5a that elicit a plethora of physiological responses that range from chemoattraction to apoptosis. Initially, complement was thought to play a major role in innate immunity where a robust and rapid response is mounted against invading pathogens [4].

Recently it is becoming increasingly evident that complement also plays an important role in adaptive immunity involving T and B cells that help in elimination of pathogens [5]. One of the early studies demonstrating involvement of complement in adaptive immunity showed that the fifth component of the complement cascade, C5a, is capable of potentiating antigen- and alloantigen-induced T cell proliferative responses. It was found that the carboxyterminal arginine of C5a is not essential in order for C5a to enhance immune responses. C5ades Arg was found to augment the immune response to the level of C5a-mediated enhancement. The serum carboxypeptidase inhibitor, 2-mercaptomethyl-5-quanodinopentanoic acid, which prevents cleavage of the terminal arginine, allowed for assessment of the effects of C5a on in vitro immune responses in the presence of serum. It was shown that helper T cells are involved in C5a-mediated immuno-potentiation. Substitution of T cells by soluble T cell-replacing factors, (Fc)TRF, rendered lymphocyte cultures refractory to the enhancing properties of C5a [6].

In another study, flow cytometry analysis was used identify the complement 5a receptor (C5aR) on T cells. It was found that this is expressed at a low basal level on unstimulated T cells and was strikingly up-regulated upon PHA stimulation in a time- and dose-dependent manner. CD3+ sorted T cells as well as Jurkat T cells were shown to express C5aR mRNA as assessed by RT-PCR. In order for the scientists to demonstrate that C5a was biologically active on T cells, we investigated the chemotactic activity of C5a and observed that purified CD3+ T cells are chemotactic to C5a at nanomolar concentrations. Finally, using a combination of in situ hybridization and immunohistochemistry, the investigators showed that the T cells infiltrating the central nervous system during experimental allergic encephalomyelitis express the C5aR mRNA. These data suggest that innate inflammation may trigger T cell chemotaxis to areas of immunological need [7]. Complement components other than C5 are also involved in T cell activation. For example, in one study, allospecific immunoglobulin (Ig)G response was markedly impaired in C3- and C4-, but not in C5-deficient mice. This defect was most pronounced for second set responses. C3-deficient mice also demonstrated a decreased range of IgG isotypes. In contrast, there was no impairment of the allospecific IgM response. In functional T cell assays, the proliferative response and interferon-gamma secretion of recipient lymphocytes restimulated in vitro with donor antigen was decreased two- to threefold in C3-deficient mice [8].

The role of complement in host T cell mediated defenses also appears relevant. Indeed, patients with complement genetic deficiencies are known to possess weaker T cell responses. In animals, a strong basic research study examined the CD8(+) T cell response in influenza type A virus-infected mice treated with a peptide antagonist to C5aR to test the potential role of complement components in CD8(+) T cell responses. It was demonstrated both the frequency and absolute numbers of flu-specific CD8(+) T cells are greatly reduced in C5aR antagonist-treated mice compared with untreated mice. This reduction in flu-specific CD8(+) T cells is accompanied by attenuated antiviral cytolytic activity in the lungs. These results demonstrate that the binding of the C5a component of complement to the C5a receptor plays an important role in CD8(+) T cell responses [9]. While the previous study demonstrated reduction in complement can compromise T cell immunity, another study demonstrated enhancement of complement augmented T cell responses. The investigators used mice deficient for decay accelerating factor (DAF), which breaks down complement. Compared with wild-type mice, DAF knockout (Daf-1(−/−)) mice had markedly increased expansion in the spleen of total and viral Ag-specific CD8+ T cells after acute or chronic LCMV infection. Splenocytes from LCMV-infected Daf-1(−/−) mice also displayed significantly higher killing activity than cells from wild-type mice toward viral Ag-loaded target cells, and Daf-1(−/−) mice cleared LCMV more efficiently. Importantly, deletion of the complement protein C3 or the receptor for the anaphylatoxin C5a (C5aR) from Daf-1(−/−) mice reversed the enhanced CD8+ T cell immunity phenotype. These results demonstrate that DAF is an important regulator of CD8+ T cell immunity in viral infection and that it fulfills this role by acting as a complement inhibitor to prevent virus-triggered complement activation and C5aR signaling [10]. Others studies have confirmed a role for various complement components in manipulation of T cell immunity [11-34].

The interaction between the innate and adaptive branches of the immune system have been previously described at several levels. For example, T cell activation of dendritic cells usually requires dendritic cells to mature in order to allow for proper antigen presentation and formation of the immunological synapse [35]. It is established that immature dendritic cells are generally tolerogenic, and induce T regulatory cells as opposed to proper T cell activation [36-82]. The process of immature dendritic cells stimulating suppressor T cells is well known in cancer, in which tumors inhibit dendritic cell maturation through production of factors such as VEGF, PGE-2, IL-10 and TGF-beta [83-86]. In the natural context, apoptotic cells possess phosphotidylserine on their surface, which maintains dendritic cells in immature states [87-98]. In contrast, during tissue damage, or infection, dendritic cells mature due to activation of receptors such as toll like receptors. Mature dendritic cells subsequent activate T cell immunity due to expression of both Signal 1 (MHC/antigen) and Signal 2 (costimulatory signals) [99]. Interestingly, some studies have shown that apoptotic bodies actually inhibit expression and/or signaling of toll like receptors [100-103].

At a basic level, complement activation is known to occur through three different pathways: alternate, classical, and lectin, involving proteins that mostly exist as inactive zymogens that are then sequentially cleaved and activated. All pathways of complement activation lead to cleavage of the C5 molecule generating the anaphylatoxin C5a and, C5b that subsequently forms the terminal complement complex (C5b-9). C5a exerts a predominant pro-inflammatory activity through interactions with the classical G-protein coupled receptor C5aR (CD88) as well as with the non-G protein coupled receptor C5L2 (GPR77), expressed on various immune and non-immune cells. C5b-9 causes cytolysis through the formation of the membrane attack complex (MAC), and sub-lytic MAC and soluble C5b-9 also possess a multitude of non-cytolytic immune functions. These two complement effectors, C5a and C5b-9, generated from C5 cleavage, are key components of the complement system responsible for propagating and/or initiating pathology in different diseases, including paroxysmal nocturnal hemoglobinuria, rheumatoid arthritis, ischemia-reperfusion injuries and neurodegenerative diseases.

The role of the DC in vivo may be conceptualized in a very general sense as a dual purpose cell: In conditions of homeostasis, DC reside in an immature state and promote tolerance, in contrast, when DC are exposed to injury/damage signals they mature and induce T cell activation. In the context of the invention the utilization of gene therapy may be performed to inhibit dendritic cell maturation in order to promote tolerogenesis in the situations of autoimmune endometrial atrophy. This general paradigm can be observed in the four conditions of tolerogenesis that will be discussed in the specification, particularly pregnancy, cancer, ACAID, and oral tolerance. One of skill in the art will utilize these conditions of natural tolerogenesis to guide the use of mesenchymal stem cells are promoters of the tolerogenic process. The direct use of mesenchymal stem cells as a “reprogrammer” of the immune system via intralymphatic or perilymphatic administration has not been previously contemplated, due to the general thought in the art that this cell population is primarily of a regenerative nature. In pregnancy circulating factors such as TGF-b family members [104] and hCG [105], have been reported to inhibit DC maturation and function [106, 107]. DC with tolerogenic properties are found at the maternal-fetal interface and express high concentrations of the immune suppressive enzyme indolamine 2,3 deoxygenase (IDO). Through local tryptophan depletion, as well as production of immune suppressive metabolites, cells expressing IDO have been demonstrated to induce T cell apoptosis, and more recently to elicit generation of T regulatory (Treg) cells [108, 109]. The critical role of this enzyme in pregnancy can be seen in studies where IDO inhibition results in immunologically mediated spontaneous abortion [110]. Accordingly, it is within the scope of the current invention to manipulate in vivo conditions using mesenchymal stem cells so as to generate a tolerance promoting environment similar to that which occurs in conditions of natural tolerogenesis. Particularly, in one embodiment, mesenchymal stem cells are administered together with a physiological concentration of hCG to elicit tolerogenesis. Administration of the mesenchymal stem cells, and/or of the hCG may be intravenous, intralymphatic, or perilymphatic. In another embodiment, mesenchymal stem cells are administered together with TGF-beta to elicit tolerogeneisis. In another embodiment mesenchymal stem cells are administered together with IDO gene therapy to promote tolerogenesis Inhibition of DC maturation and/or reprogramming by the tumor microenvironment has been well documented in numerous clinical system and animal experiments. DC isolated from tumor draining lymph nodes in melanoma [111, 112], ovarian [113], breast [114], and lung cancer [115] have been characterized as having an immature/plasmacytoid phenotype, suppressed T cell activating ability and possess elevated levels of IDO. Manipulation of DC by silencing the gene IDO using siRNA has been demonstrated to evoke productive T cell immunity towards melanoma [116]. Secretion of VEGF by tumor cells is one of several proposed mechanisms for increased immature DC in tumor patients [117]. Administration of the anti-VEGFR antibody bevacizumab in patients with a variety of tumors was demonstrated to increase DC maturation and restore T cell activating activity [118]. Accordingly, within the context of the current invention, mesenchymal stem cells may be administered together with concentrations of VEGF found in the tumor to be tolerogenic.

The gene therapeutic may be delivered using a variety of routes from intrauterine, direct injection with or without ultrasound guidance or systemic based on the clinical scenario of the patient. The uterine tissue may need to be pretreated with the gene therapeutic to optimize the receptibility of the fertilized egg. A pretreatment of the uterine lining may be required to optimize the gene therapeutic or to increase retention and survival using the carrier solution of platelet containing plasma with low hematocrit or, platelet lysate, either fresh or reconstituted lyophilized platelet lysate. The gene therapy described in the invention can be further increased in ability to cure damaged endometrium by addition of various Examples of anti-inflammatory agents include, but are not limited to, a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-.alpha. release inhibitor), thalidomide analogues (which reduce TNF-.alpha. production by macrophages), bone morphogenetic protein (BMP) type 2 or BMP-4 (inhibitors of caspase 8, a TNF-.alpha. activator), quinapril (an inhibitor of angiotensin II, which upregulates TNF-.alpha.), interferons such as IL-11 (which modulate TNF-.alpha. receptor expression), and aurin-tricarboxylic acid (which inhibits TNF-.alpha.), guanidinoethyldisulfide, or a combination thereof. Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, 1, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone, or a combination thereof. Additionally, one or more steroids may be administered within the context of the invention. Exemplary steroids include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

### REFERENCES

ADDIN EN.REFLIST 1. Walport, M. J., Complement. First of two parts. N Engl J Med, 2001. 344(14): p. 1058-66.
2. Snyderman, R., H. Gewurz, and S. E. Mergenhagen, Interactions of the complement system with endotoxic lipopolysaccharide. Generation of a factor chemotactic for polymorphonuclear leukocytes. J Exp Med, 1968. 128(2): p. 259-75.
3. Haddad, A. and A. M. Wilson, Biochemistry, Complement, in StatPearls. 2020: Treasure Island (FL).
4. Lehnardt, S., Innate immunity and neuroinflammation in the CNS: the role of microglia in Toll-like receptor-mediated neuronal injury. Glia, 2010. 58(3): p. 253-63.
5. Dunkelberger, J. R. and W. C. Song, Complement and its role in innate and adaptive immune responses. Cell Res, 2010. 20(1): p. 34-50.
6. Morgan, E. L., et al., Anaphylatoxin-mediated regulation of the immune response. II. C5a-mediated enhancement of human humoral and T cell-mediated immune responses. J Immunol, 1983. 130(3): p. 1257-61.
7. Nataf, S., et al., Human T cells express the C5a receptor and are chemo attracted to C5a. J Immunol, 1999. 162(7): p. 4018-23.
8. Marsh, J. E., et al., The allogeneic T and B cell response is strongly dependent on complement components C3 and C4. Transplantation, 2001. 72(7): p. 1310-8.
9. Kim, A. H., et al., Complement C5a receptor is essential for the optimal generation of antiviral CD8+ T cell responses. J Immunol, 2004. 173(4): p. 2524-9.
10. Fang, C., et al., Complement-dependent enhancement of CD8+ T cell immunity to lymphocytic choriomeningitis virus infection in decay-accelerating factor-deficient mice. J Immunol, 2007. 179(5): p. 3178-86.
11. Wang, H., et al., Inhibition of terminal complement components in presensitized transplant recipients prevents antibody-mediated rejection leading to long-term graft survival and accommodation. J Immunol, 2007. 179(7): p. 4451-63.
12. Lalli, P. N., et al., Decay accelerating factor can control T cell differentiation into IFN-gamma-producing effector cells via regulating local C5a-induced IL-12 production. J Immunol, 2007. 179(9): p. 5793-802.
13. Strainic, M. G., et al., Locally produced complement fragments C5a and C3a provide both costimulatory and survival signals to naive CD4+ T cells. Immunity, 2008. 28(3): p. 425-35.
14. Liu, J., et al., IFN-gamma and IL-17 production in experimental autoimmune encephalomyelitis depends on local APC-T cell complement production. J Immunol, 2008. 180(9): p. 5882-9.
15. Hegde, G. V., et al., A conformationally-biased, response-selective agonist of C5a acts as a molecular adjuvant by modulating antigen processing and presentation activities of human dendritic cells. Int Immunopharmacol, 2008. 8(6): p. 819-27.
16. Lalli, P. N., et al., Locally produced C5a binds to T cell-expressed C5aR to enhance effector T-cell expansion by limiting antigen-induced apoptosis. Blood, 2008. 112(5): p. 1759-66.
17. Pavlov, V., et al., Donor deficiency of decay-accelerating factor accelerates murine T cell-mediated cardiac allograft rejection. J Immunol, 2008. 181(7): p. 4580-9.
18. Fang, C., et al., Complement promotes the development of inflammatory T-helper 17 cells through synergistic interaction with Toll-like receptor signaling and interleukin-6 production. Blood, 2009. 114(5): p. 1005-15.
19. Raedler, H., et al., Primed CD8(+) T-cell responses to allogeneic endothelial cells are controlled by local complement activation. Am J Transplant, 2009. 9(8): p. 1784-95.
20. Li, Q., et al., The complement inhibitor FUT-175 suppresses T cell autoreactivity in experimental autoimmune encephalomyelitis. Am J Pathol, 2009. 175(2): p. 661-7.
21. Li, Q., et al., Augmenting DAF levels in vivo ameliorates experimental autoimmune encephalomyelitis. Mol Immunol, 2009. 46(15): p. 2885-91.
22. Xu, R., et al., Complement C5a regulates IL-17 by affecting the crosstalk between DC and gammadelta T cells in CLP-induced sepsis. Eur J Immunol, 2010. 40(4): p. 1079-88.
23. Hashimoto, M., et al., Complement drives Th17 cell differentiation and triggers autoimmune arthritis. J Exp Med, 2010. 207(6): p. 1135-43.
24. Chen, G., et al., Blockade of complement activation product C5a activity using specific antibody attenuates intestinal damage in trinitrobenzene sulfonic acid induced model of colitis. Lab Invest, 2011. 91(3): p. 472-83.
25. Han, G., et al., gammadeltaT-cell function in sepsis is modulated by C5a receptor signalling. Immunology, 2011. 133(3): p. 340-9.
26. Raedler, H., et al., Anti-complement component C5 mAb synergizes with CTLA4Ig to inhibit alloreactive T cells and prolong cardiac allograft survival in mice. Am J Transplant, 2011. 11(7): p. 1397-406.
27. Vieyra, M., et al., Complement regulates CD4 T-cell help to CD8 T cells required for murine allograft rejection. Am J Pathol, 2011. 179(2): p. 766-74.
28. Fusakio, M. E., et al., C5a regulates NKT and NK cell functions in sepsis. J Immunol, 2011. 187(11): p. 5805-12.
29. Mashruwala, M. A., et al., A defect in the synthesis of Interferon-gamma by the T cells of Complement-C5 deficient mice leads to enhanced susceptibility for tuberculosis. Tuberculosis (Edinb), 2011. 91 Suppl 1: p. S82-9.
30. Strainic, M. G., et al., Absence of signaling into CD4(+) cells via C3aR and C5aR enables autoinductive TGF-beta1 signaling and induction of Foxp3(+) regulatory T cells. Nat Immunol, 2013. 14(2): p. 162-71.
31. Liu, T., et al., An essential role for C5aR signaling in the optimal induction of a malaria-specific CD4+ T cell response by a whole-killed blood-stage vaccine. J Immunol, 2013. 191(1): p. 178-86.
32. Ma, N., et al., C5a regulates IL-12+DC migration to induce pathogenic Th1 and Th17 cells in sepsis. PLoS One, 2013. 8(7): p. e69779.
33. Cravedi, P., et al., Immune cell-derived C3a and C5a costimulate human T cell alloimmunity. Am J Transplant, 2013. 13(10): p. 2530-9.
34. Yamanaka, K., et al., Depression of Complement Regulatory Factors in Rat and Human Renal Grafts Is Associated with the Progress of Acute T-Cell Mediated Rejection. PLoS One, 2016. 11(2): p. e0148881.
35. Steinman, R. M., Dendritic cells: understanding immunogenicity. Eur J Immunol, 2007. 37 Suppl 1: p. 553-60.
36. Levings, M. K., et al., Differentiation of Tr1 cells by immature dendritic cells requires IL-10 but not CD25+CD4+ Tr cells. Blood, 2005. 105(3): p. 1162-9.
37. Ghiringhelli, F., et al., Tumor cells convert immature myeloid dendritic cells into TGF-beta-secreting cells inducing CD4+CD25+ regulatory T cell proliferation. J Exp Med, 2005. 202(7): p. 919-29.
38. Stepkowski, S. M., et al., Immature syngeneic dendritic cells potentiate tolerance to pancreatic islet allografts depleted of donor dendritic cells in microgravity culture condition. Transplantation, 2006. 82(12): p. 1756-63.
39. Jin, Y., et al., Induction of auto-reactive regulatory T cells by stimulation with immature autologous dendritic cells. Immunol Invest, 2007. 36(2): p. 213-32.
40. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.
41. Ureta, G., et al., Generation of dendritic cells with regulatory properties. Transplant Proc, 2007. 39(3): p. 633-7.
42. Yamazaki, S., et al., Dendritic cells are specialized accessory cells along with TGF-for the differentiation of Foxp3+CD4+ regulatory T cells from peripheral Foxp3 precursors. Blood, 2007. 110(13): p. 4293-302.
43. Gaudreau, S., et al., Granulocyte-macrophage colony-stimulating factor prevents diabetes development in NOD mice by inducing tolerogenic dendritic cells that sustain the suppressive function of CD4+CD25+ regulatory T cells. J Immunol, 2007. 179(6): p. 3638-47.
44. Wu, K., et al., Suppression of allergic inflammation by allergen-DNA-modified dendritic cells depends on the induction of Foxp3+ Regulatory T cells. Scand J Immunol, 2008. 67(2): p. 140-51.
45. Sarris, M., et al., Neuropilin-1 expression on regulatory T cells enhances their interactions with dendritic cells during antigen recognition. Immunity, 2008. 28(3): p. 402-13.
46. Zhang, X., et al., Generation of therapeutic dendritic cells and regulatory T cells for preventing allogeneic cardiac graft rejection. Clin Immunol, 2008. 127(3): p. 313-21.
47. Cools, N., et al., Immunosuppression induced by immature dendritic cells is mediated by TGF-beta/IL-10 double-positive CD4+ regulatory T cells. J Cell Mol Med, 2008. 12(2): p. 690-700.
48. Wang, L., et al., Programmed death 1 ligand signaling regulates the generation of adaptive Foxp3+CD4+ regulatory T cells. Proc Natl Acad Sci USA, 2008. 105(27): p. 9331-6.
49. Marguti, I., et al., Expansion of CD4+CD25+Foxp3+ T cells by bone marrow-derived dendritic cells. Immunology, 2009. 127(1): p. 50-61.
50. Qadura, M., et al., Reduction of the immune response to factor VIII mediated through tolerogenic factor VIII presentation by immature dendritic cells. J Thromb Haemost, 2008. 6(12): p. 2095-104.
51. Kuo, Y. R., et al., Alloantigen-pulsed host dendritic cells induce T-cell regulation and prolong allograft survival in a rat model of hindlimb allotransplantation. J Surg Res, 2009. 153(2): p. 317-25.
52. Bamboat, Z. M., et al., Human liver dendritic cells promote T cell hyporesponsiveness. J Immunol, 2009. 182(4): p. 1901-11.
53. Jin, C. J., et al., All-trans retinoic acid inhibits the differentiation, maturation, and function of human monocyte-derived dendritic cells. Leuk Res, 2010. 34(4): p. 513-20.
54. Zahorchak, A. F., G. Raimondi, and A. W. Thomson, Rhesus monkey immature monocyte-derived dendritic cells generate alloantigen-specific regulatory T cells from circulating CD4+CD127−/lo T cells. Transplantation, 2009. 88(9): p. 1057-64.
55. Kushwah, R., et al., Apoptotic dendritic cells induce tolerance in mice through suppression of dendritic cell maturation and induction of antigen-specific regulatory T cells. J Immunol, 2009. 183(11): p. 7104-18.
56. Dai, H., et al., Programmed death-1 signaling is essential for the skin allograft protection by alternatively activated dendritic cell infusion in mice. Transplantation, 2009. 88(7): p. 864-73.
57. Wang, L., Adaptive Treg generation by DCs and their functional analysis. Methods Mol Biol, 2010. 595: p. 403-12.
58. Schildknecht, A., et al., FoxP3+ regulatory T cells essentially contribute to peripheral CD8+ T-cell tolerance induced by steady-state dendritic cells. Proc Natl Acad Sci USA, 2010. 107(1): p. 199-203.
59. Heng, Y., et al., Adoptive transfer of FTY720-treated immature BMDCs significantly prolonged cardiac allograft survival. Transpl Int, 2010. 23(12): p. 1259-70.
60. Muller, T., et al., Iloprost has potent anti-inflammatory properties on human monocyte-derived dendritic cells. Clin Exp Allergy, 2010. 40(8): p. 1214-21.
61. Kong, W., J. H. Yen, and D. Ganea, Docosahexaenoic acid prevents dendritic cell maturation, inhibits antigen-specific Th1/Th17 differentiation and suppresses experimental autoimmune encephalomyelitis. Brain Behav Immun, 2011. 25(5): p. 872-82.
62. Takeda, M., et al., Oral administration of an active form of vitamin D3 (calcitriol) decreases atherosclerosis in mice by inducing regulatory T cells and immature dendritic cells with tolerogenic functions. Arterioscler Thromb Vase Biol, 2010. 30(12): p. 2495-503.
63. Stary, G., et al., Glucocorticosteroids modify Langerhans cells to produce TGF-beta and expand regulatory T cells. J Immunol, 2011. 186(1): p. 103-12.
64. Pletinckx, K., et al., Role of dendritic cell maturity/costimulation for generation, homeostasis, and suppressive activity of regulatory T cells. Front Immunol, 2011. 2: p. 39.
65. Wolfle, S. J., et al., PD-L1 expression on tolerogenic APCs is controlled by STAT-3. Eur J Immunol, 2011. 41(2): p. 413-24.
66. Schuler, P. J., et al., Dendritic cell generation and CD4+ CD25high FOXP3+ regulatory t cells in human head and neck carcinoma during radio-chemotherapy. Eur J Med Res, 2011. 16(2): p. 57-62.
67. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
68. Amaral, M. M., et al., Thioperamide induces CD4 CD25 Foxp3 regulatory T lymphocytes in the lung mucosa of allergic mice through its action on dendritic cells. J Asthma Allergy, 2011. 4: p. 93-102.
69. Presicce, P., et al., Myeloid dendritic cells isolated from tissues of SIV-infected Rhesus macaques promote the induction of regulatory T cells. AIDS, 2012. 26(3): p. 263-73.
70. Wang, G. Y., et al., Rapamycin combined with donor immature dendritic cells promotes liver allograft survival in association with CD4(+) CD25(+) Foxp3(+) regulatory T cell expansion. Hepatol Res, 2012. 42(2): p. 192-202.
71. Petzold, C., et al., Targeted antigen delivery to DEC-205(+) dendritic cells for tolerogenic vaccination. Rev Diabet Stud, 2012. 9(4): p. 305-18.
72. Wang, G. Y., et al., Rapamycin combined with allogenic immature dendritic cells selectively expands CD4+CD25+Foxp3+ regulatory T cells in rats. Hepatobiliary Pancreat Dis Int, 2012. 11(2): p. 203-8.
73. Zhou, F., et al., Immune tolerance induced by intravenous transfer of immature dendritic cells via up-regulating numbers of suppressive IL-10(+) IFN-gamma(+)-producing CD4(+) T cells. Immunol Res, 2013. 56(1): p. 1-8.
74. Volchenkov, R., et al., Type 1 regulatory T cells and regulatory B cells induced by tolerogenic dendritic cells. Scand J Immunol, 2013. 77(4): p. 246-54.
75. Zhang, G., et al., Triptolide-conditioned dendritic cells induce allospecific T-cell regulation and prolong renal graft survival. J Invest Surg, 2013. 26(4): p. 191-9.
76. Farias, A. S., et al., Vitamin D3 induces IDO+ tolerogenic DCs and enhances Treg, reducing the severity of EAE. CNS Neurosci Ther, 2013. 19(4): p. 269-77.
77. Gao, X. W., et al., Mechanism of immune tolerance induced by donor derived immature dendritic cells in rat high-risk corneal transplantation. Int J Ophthalmol, 2013. 6(3): p. 269-75.
78. Lindenberg, J. J., et al., IL-10 conditioning of human skin affects the distribution of migratory dendritic cell subsets and functional T cell differentiation. PLoS One, 2013. 8(7): p. e70237.
79. Huang, Y., et al., Increased expression of herpesvirus entry mediator in 1,25-dihydroxyvitamin D3-treated mouse bone marrow-derived dendritic cells promotes the generation of CD4(+)CD25(+)Foxp3(+) regulatory T cells. Mol Med Rep, 2014. 9(3): p. 813-8.
80. Pletinckx, K., et al., Immature dendritic cells convert anergic nonregulatory T cells into Foxp3-IL-10+ regulatory T cells by engaging CD28 and CTLA-4. Eur J Immunol, 2015. 45(2): p. 480-91.
81. Wei, Y., et al., Infusion of dendritic cells carrying donor lymphocytes treated with 8-methoxypsoralen and ultraviolet A light induces CD19+ IL-10+ regulatory B cells and promotes skin allograft survival. Transplant Proc, 2014. 46(10): p. 3641-6.
82. Dong, M., et al., Rapamycin Combined with Immature Dendritic Cells Attenuates Obliterative Bronchiolitis in Trachea Allograft Rats by Regulating the Balance of Regulatory and Effector T Cells. Int Arch Allergy Immunol, 2015. 167(3): p. 177-85.
83. Bergmann, C., et al., Expansion and characteristics of human T regulatory type 1 cells in co-cultures simulating tumor microenvironment. Cancer Immunol Immunother, 2007. 56(9): p. 1429-42.
84. Kaporis, H. G., et al., Human basal cell carcinoma is associated with Foxp3+ T cells in a Th2 dominant microenvironment. J Invest Dermatol, 2007. 127(10): p. 2391-8.
85. Koido, S., et al., In vitro generation of cytotoxic and regulatory T cells by fusions of human dendritic cells and hepatocellular carcinoma cells. J Transl Med, 2008. 6: p. 51.
86. Li, L., et al., Hepatoma cells inhibit the differentiation and maturation of dendritic cells and increase the production of regulatory T cells. Immunol Lett, 2007. 114(1): p. 38-45.
87. Shi, D., et al., Artificial phosphatidylserine liposome mimics apoptotic cells in inhibiting maturation and immunostimulatory function of murine myeloid dendritic cells in response to 1-chloro-2,4-dinitrobenze in vitro. Arch Dermatol Res, 2007. 299(7): p. 327-36.
88. Kranich, J., et al., Follicular dendritic cells control engulfment of apoptotic bodies by secreting Mfge8. J Exp Med, 2008. 205(6): p. 1293-302.
89. Rodriguez-Manzanet, R., et al., T and B cell hyperactivity and autoimmunity associated with niche-specific defects in apoptotic body clearance in TIM-4-deficient mice. Proc Natl Acad Sci USA, 2010. 107(19): p. 8706-11.
90. Frey, B. and U.S. Gaipl, The immune functions of phosphatidylserine in membranes of dying cells and microvesicles. Semin Immunopathol, 2011. 33(5): p. 497-516.
91. Saas, P., et al., Phosphatidylserine-expressing cell by-products in transfusion: A pro-inflammatory or an anti-inflammatory effect? Transfus Clin Biol, 2012. 19(3): p. 90-7.
92. Trahtemberg, U. and D. Mevorach, Apoptotic Cells Induced Signaling for Immune Homeostasis in Macrophages and Dendritic Cells. Front Immunol, 2017. 8: p. 1356.
93. Perruche, S., et al., CD3-specific antibody-induced immune tolerance involves transforming growth factor-beta from phagocytes digesting apoptotic T cells. Nat Med, 2008. 14(5): p. 528-35.
94. Dumitriu, I. E., et al., Human dendritic cells produce TGF-beta 1 under the influence of lung carcinoma cells and prime the differentiation of CD4+CD25+Foxp3+ regulatory T cells. J Immunol, 2009. 182(5): p. 2795-807.
95. Ragni, M. V., et al., Factor VIII-pulsed dendritic cells reduce anti-factor VIII antibody formation in the hemophilia A mouse model. Exp Hematol, 2009. 37(6): p. 744-54.
96. Kushwah, R., et al., Uptake of apoptotic DC converts immature DC into tolerogenic DC that induce differentiation of Foxp3+ Treg. Eur J Immunol, 2010. 40(4): p. 1022-35.
97. Zheng, D. H., et al., Uptake of donor lymphocytes treated with 8-methoxypsoralen and ultraviolet A light by recipient dendritic cells induces CD4+CD25+Foxp3+ regulatory T cells and down-regulates cardiac allograft rejection. Biochem Biophys Res Commun, 2010. 395(4): p. 540-6.
98. Carrascal, M. A., et al., Sialyl Tn-expressing bladder cancer cells induce a tolerogenic phenotype in innate and adaptive immune cells. Mol Oncol, 2014. 8(3): p. 753-65.
99. Lutz, M. B., Induction of CD4(+) Regulatory and Polarized Effector/helper T Cells by Dendritic Cells. Immune Netw, 2016. 16(1): p. 13-25.
100. Verbovetski, I., et al., Opsonization of apoptotic cells by autologous iC3b facilitates clearance by immature dendritic cells, down-regulates DR and CD86, and up-regulates CC chemokine receptor 7. J Exp Med, 2002. 196(12): p. 1553-61.
101. Fadok, V. A., et al., Macrophages that have ingested apoptotic cells in vitro inhibit proinflammatory cytokine production through autocrine/paracrine mechanisms involving TGF-beta, PGE2, and PAF. J Clin Invest, 1998. 101(4): p. 890-8.
102. McDonald, P. P., et al., Transcriptional and translational regulation of inflammatory mediator production by endogenous TGF-beta in macrophages that have ingested apoptotic cells. J Immunol, 1999. 163(11): p. 6164-72.
103. Stuart, L. M., et al., Inhibitory effects of apoptotic cell ingestion upon endotoxin-driven myeloid dendritic cell maturation. J Immunol, 2002. 168(4): p. 1627-35.
104. Segerer, S. E., et al., The glycoprotein-hormones activin A and inhibin A interfere with dendritic cell maturation. Reprod Biol Endocrinol, 2008. 6: p. 17.
105. Segerer, S. E., et al., Impact of female sex hormones on the maturation and function of human dendritic cells. Am J Reprod Immunol, 2009. 62(3): p. 165-73.
106. Shojaeian, J., et al., Immunosuppressive effect of pregnant mouse serum on allostimulatory activity of dendritic cells. J Reprod Immunol, 2007. 75(1): p. 23-31.
107. Zarnani, A. H., et al., Microenvironment of the feto-maternal interface protects the semiallogenic fetus through its immunomodulatory activity on dendritic cells. Fertil Steril, 2008. 90(3): p. 781-8.
108. Jurgens, B., et al., Interferon-gamma-triggered indoleamine 2,3-dioxygenase competence in human monocyte-derived dendritic cells induces regulatory activity in allogeneic T cells. Blood, 2009. 114(15): p. 3235-43.
109. Brenk, M., et al., Tryptophan deprivation induces inhibitory receptors ILT3 and ILT4 on dendritic cells favoring the induction of human CD4+CD25+Foxp3+ T regulatory cells. J Immunol, 2009. 183(1): p. 145-54.
110. Mellor, A. L., et al., Prevention of T cell-driven complement activation and inflammation by tryptophan catabolism during pregnancy. Nat Immunol, 2001. 2(1): p. 64-8.
111. Lee, J. R., et al., Pattern of recruitment of immunoregulatory antigen-presenting cells in malignant melanoma. Lab Invest, 2003. 83(10): p. 1457-66.
112. Botella-Estrada, R., et al., Cytokine expression and dendritic cell density in melanoma sentinel nodes. Melanoma Res, 2005. 15(2): p. 99-106.
113. Curiel, T. J., et al., Blockade of B7-H1 improves myeloid dendritic cell-mediated antitumor immunity. Nat Med, 2003. 9(5): p. 562-7.
114. Almand, B., et al., Clinical significance of defective dendritic cell differentiation in cancer. Clin Cancer Res, 2000. 6(5): p. 1755-66.
115. Almand, B., et al., Increased production of immature myeloid cells in cancer patients: a mechanism of immunosuppression in cancer. J Immunol, 2001. 166(1): p. 678-89.
116. Zheng, X., et al., Reinstalling antitumor immunity by inhibiting tumor-derived immunosuppressive molecule IDO through RNA interference. J Immunol, 2006. 177(8): p. 5639-46.
117. Johnson, B., et al., Physiology and therapeutics of vascular endothelial growth factor in tumor immunosuppression. Curr Mol Med, 2009. 9(6): p. 702-7.
118. Osada, T., et al., The effect of anti-VEGF therapy on immature myeloid cell and dendritic cells in cancer patients. Cancer Immunol Immunother, 2008. 57(8): p. 1115-24.

1. A method of preventing and/or treating endometrial atrophy, comprising the steps of: a) selecting a patient in need of treatment; b) administering an effective amount of a gene therapeutic to the patient need of treatment; c) assessing effect of said gene therapeutic infusion and adjusting concentration and frequency based on response.

2. The method of claim 1, wherein said gene therapeutic is administered into the ovarian artery.

3. The method of claim 2, wherein said gene therapeutic is administered in the form of free DNA plasma.

4. The method of claim 2, wherein said gene therapeutic is administered in the form of free mRNA.

5. The method of claim 2, wherein said gene therapeutic is administered in the form of an adenoviral vector.

6. The method of claim 2, wherein said gene therapeutic is administered in the form of a lentiviral vector.

7. The method of claim 2, wherein said gene therapeutic is administered in the form of a cellular therapy.

8. The method of claim 2, wherein said gene therapeutic is administered in the form of an exosomal therapy.

9. The method of claim 1, wherein said gene therapeutic is angiogenic.

10. The method of claim 9, wherein said angiogenic gene is VEGF.

11. The method of claim 9, wherein said angiogenic gene is SDF-1.

12. The method of claim 1, wherein said gene therapeutic is anti-apoptotic.

13. The method of claim 12, wherein said anti-apoptotic gene is survivin.

14. The method of claim 12, wherein said anti-apoptotic gene is interleukin-10.

15. The method of claim 1, wherein said gene therapeutic is anti-fibrotic.

16. The method of claim 15, wherein said anti-fibrotic gene is antisense to TGF-beta.

17. The method of claim 15, wherein said anti-fibrotic gene is MMP1.

18. The method of claim 1, wherein said gene therapeutic are directly injected into the uterine lining with or without ultrasound guidance.

19. The method of claim 1, wherein said gene therapeutic are directly injected into the uterine lining via transvaginal approach.

20. The method of claim 1, wherein said gene therapeutic are placed in carrier solution of reconstituted lyophilized or fresh platelet lysate for injection.
